(19) United States
(12) Patent Application Publication
(10) Pub. No.: US 2015/0305956 A1
(43) Pub. Date: Oct. 29, 2015

(54) Urine Collector

(71) Applicant: Sophia Hai Yun SU, North Point (HK)

(72) Inventor: Sophia Hai Yun SU, North Point (HK)

(21) Appl. No.: 14/670,399

(22) Filed: Mar. 26, 2015

(30) Foreign Application Priority Data
Apr. 29, 2014 (HK) 14104119.2
Jun. 11, 2014 (CN) 201420309147.4

(51) Int. Cl. A61G 9/00; A61F 5/44; A61F 5/455

(52) U.S. Cl. CPC A61G 9/006; A61F 5/455; A61F 5/4404

(57) ABSTRACT

A urine collector for collecting urine from a user includes a collector, a top cover coupled on top of the collector, and a urine guider. The collector has a bottom wall and a surrounding wall upwardly extended therefrom to define a cavity. The top cover has a ring shape and defines a top side with a size adapted for covering a hip of the user, wherein the top side of the top cover is sloped downwardly from an outer peripheral edge of the top cover to an inner peripheral edge thereof for guiding urine flowing into the collector. The urine guider is provided at a bottom portion of the collector for guiding urine at the urine guider within the cavity of the collector, wherein the collector is not limited to urinate portion but supporting and covering the entire hip area of the user to prevent any leak out of the urine while the urine guider can also avoid any splashing and leakage.

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to a health care product, and more particularly to a urine collector for bedridden female patients.

2. Description Orelated Arts

The use of a urinary collection device is well known to collect urine from a physically-disabled person. Especially for female patients, the female urinary collection device can substantially reduce the workload for health care workers. Generally, a potty or a bed urinal is used as the urinary collection device to collect urine from the patient. However, such conventional urinary collection devices are difficult to use for urine collection and urine always leaks between the contacting surface of the urinary collection device and the body of the patient. Bed linens and clothing are contaminated due to the urine leakage, so as to cause different health problems such as infection or pressure ulcers.

An improved urine collector is a simple, comfortable to wear, effective, and convenience appliance to take care of the bedridden patients, wherein the improved urine collector can reduce workloads for nurses and families. In other words, the conventional urine collector can help the care workers and/or patient's family members to reduce the stress for caring the bedridden patients.

China Patent Publication No. ZL20102019533939, ZL200720024857.2, ZL200420053761.5, and ZL200620081221.7 disclosed an improved urine collector for bedridden female patients to solve the common problems of the current urine collectors. Since the improved urine collector can only cover on the meatus urinarius of the female patient, the improved urine collector is difficult to collect all discharging urine from the patients. As a result, bed linens and clothing are easily to be contaminated by the urine. In addition, physically-disabled female patients with low limb fracture, pelvic fracture, or postpartum are difficult to use the above mentioned urine collectors because they are not powerful enough to retain the urine collector in position. An improper use of urine collector may cause serious injures due to the excessive movement of the patients.

### SUMMARY OF THE PRESENT INVENTION

A main object of the present invention is to provide a urine collector, wherein the urine collector is formed in a compact structure and is light in weight, such that the urine collector can be used by the patients for overnight. In other words, health care workers do not need to frequently unload the urine in the urine collector of the present invention.

According to the present invention, the foregoing and other objects and advantages are attained by a urine collector, comprising a collector, a transporting tube, and a container. The transporting tube can be a PET tube, a latex tube, or a silica gel tube. The collector is pot-shaped container having a top opening, and is preferably made of hard plastic, foam material, or hard rubber. The urine collector further comprises a ring-shaped top cover coupled on a top edge of the collector, wherein the top cover is preferably made of plastic or latex, and is obliquely extended toward a center of the top cover, such that a top side of the top cover is slanted and downwardly extended toward the top opening of the collector. The urine collector further comprises a guider and a discharging unit arranged on a bottom portion of the collector, wherein a bottom edge of the guider is lower than a bottom surface of the collector. In addition, the collector has a surrounding sidewall, wherein the surrounding sidewall can be a straight wall, an obliquely and downward-extended arc-shaped wall, or an inclined straight wall. It is worth mentioning that the collector also can be made of metal or wood.

Accordingly, the discharging unit comprises a collecting port and a discharging port located on an inner wall of the transporting tube, wherein the collecting port is operatively communicated with the guider and the discharging port is operatively linked with the transporting tube. The collecting port and the discharging port are operatively connected with each other.

Accordingly, the hip portion of the patient sits on the collector that the collector will cover the urethra of the patient so as to prevent the leakage of the urine, wherein the collector serves as a temporary collector for guiding the urine flowing into the container, such that the urine collector can be used for urine without frequently discharging urine in the collector.

Accordingly, the container is not limited to a container with or without scale and a sewage drain and the like.

The present invention is simple in structure, light in weight, and easy to operate, such that the present invention has benefits for the bedridden patients for clinical care and home care, and it is also convenient for the bedridden patients to solve their urinating problems by themselves who cannot get to a restroom to urinate in a convenience and privacy manner. At the same time, the ring-shaped top cover is preferably made of latex, which is very comfortable for the user to sit on, and the ring-shaped top cover is slightly tilted towards a center thereof to ensure the discharging urine flowing into the collector. The shape of the guider and the surrounding sidewall are able to facilitate the discharging urine flowing into the collector, such that the urine can be effectively discharged from the collector to the container, so as to reduce the leakage of urine for contaminating the bedding, and further reduce the incidence of the ulcers.

Additional advantages and features of the invention will become apparent from the description which follows, and may be realized by means of the instrumentalities and combinations particular point out in the appended claims.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of a urine collector according to a preferred embodiment of the present invention.

FIG. 2 is a sectional view of the urine collector according to the above preferred embodiment of the present invention.

FIG. 3 illustrates a first alternative mode of the collector of the urine collector according to the above preferred embodiment of the present invention, illustrating the arc-shaped wall of the collector.

FIG. 4 illustrates a second alternative mode of the collector of the urine collector according to the above preferred embodiment of the present invention, illustrating the inclined wall of the collector.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

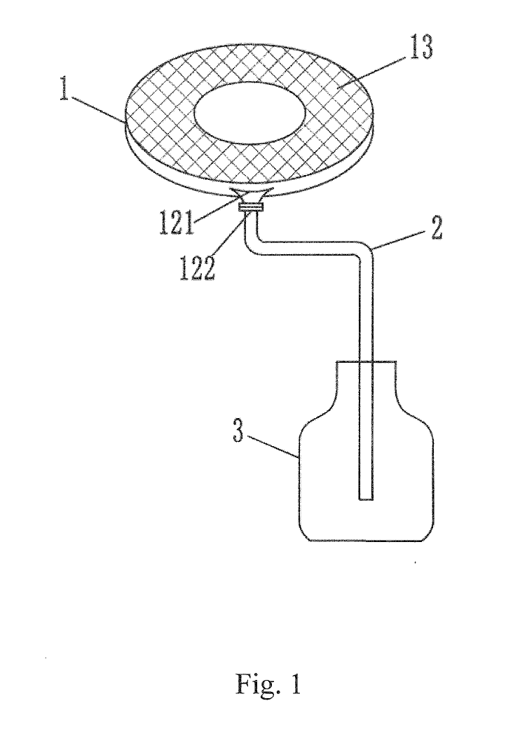

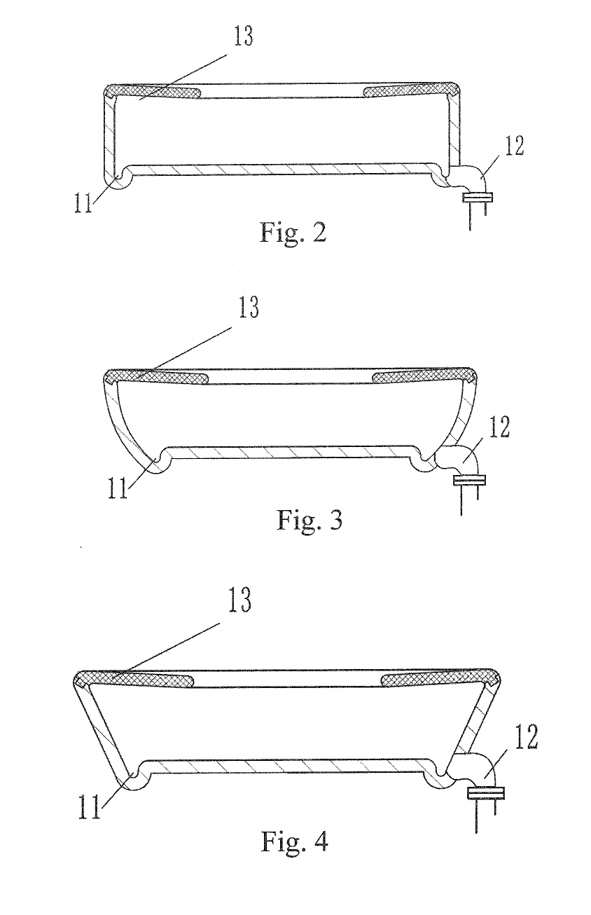

Referring to FIGS. 1 and 2 of the drawings, a urine collector according to a preferred embodiment of the present invention is illustrated, wherein the urine collector comprises a collector 1 having a top opening and a cavity, a transporting tube 2, which is made of PET material, connected to the collector 1 for transporting the urine, and a container 3 operatively connected with the transporting tube 2 for containing the urine from the collector 1 through the transporting tube 2. The collector 1 is a pot-shaped collector and made of a rigid plastic material. Accordingly, the collector 1 comprises a urine guider 11 provided at a bottom portion of the collector 1 for guiding urine at the urine guider 11 within the cavity, and a discharging unit 12 operatively connected with the urine guider 11 of the collector 1, wherein the urine guider 11 is a guiding groove indented around a peripheral portion of the bottom wall of the collector 1. It is worth mentioning that a top portion of the urine guider 11 is lower than the bottom wall of the collector 1. And, the collector 1 further comprises a surrounding wall, wherein the surrounding wall is a vertical wall extended with respect to the bottom wall of the collector 1 that the size of the top opening of the collector 1 matches the size of the bottom side of the collector 1. In other words, the urine guider 11 is formed between the surrounding wall and the bottom wall of the collector 1.

Moreover, the collector 1 further comprises a ring-shaped top cover 13 coaxially coupled on a top edge of the collector 1 to partially cover the top opening thereof, wherein the top cover 13 is preferably made of latex. A top side of the top cover 13 is inclinedly extended from an outer peripheral edge toward a center, so as to extend toward the top opening of the collector 1 to guide the urine toward the container 1 through the top opening thereof. In other words, the slope of the top side of the top cover 13 is gradually decreased from an outer edge of the top cover 13 to an inner edge thereof. Furthermore, the discharging unit 12 comprises a collecting port 121 located at the surrounding wall of the container 1 and a discharging port 122, wherein the collecting port 121 is operatively linked with the urine guider 11 and the discharging port 122 is operatively linked with the transporting tube 2, and the collecting port 121 and the discharging port 122 are operatively connected with each other. The discharging port 122 is connected to the inner wall of the transporting tube 2. In other words, the urine moves along the guider 11 to reach the collecting port 121, and passes through the collecting port 121 and the discharging port 122 respectively, so as to reach the transporting tube 2, and then the urine moves along the transporting tube 2 to be guide into the container 3.

Accordingly, the collector 1 is placed below a hip of the patient, especially female patient, wherein the collector serves as a temporary collecting station for guiding the urine to flow into the container 3.

FIG. 3 illustrates a first alternative mode the urine collector, wherein the urine collector comprises a collector 1 having a top opening and a cavity, a transporting tube 2, which is made of latex material, connected to the collector 1 for transporting the urine, and a container 3 operatively connected with the transporting tube 2 for containing the urine from the collector 1 through the transporting tube 2. The collector 1 is a pot-shaped collector and made of a rigid rubber material. Accordingly, the collector 1 comprises a urine guider 11 provided at a bottom portion of the collector 1 for guiding urine at the urine guider 11 within the cavity, and a discharging unit 12 operatively connected with the urine guider 11 of the collector 1, wherein the urine guider 11 is a guiding groove indented around a peripheral portion of the bottom wall of the collector 1. It is worth mentioning that a top portion of the urine guider 11 is lower than the bottom wall of the collector 1. And, the collector 1 further comprises a surrounding wall, wherein the surrounding wall is an arc-shaped wall extended with respect to the bottom wall of the collector 1 that the size of the top opening of the collector 1 is larger than the size of the bottom wall of the collector 1. In other words, the urine guider 11 is formed between the surrounding wall and the bottom wall of the collector 1.

Moreover, the collector 1 further comprises a ring-shaped top cover 13 coaxially coupled on a top edge of the collector 1 to partially cover the top opening thereof, wherein the top cover 13 is preferably made of latex. A top side of the top cover 13 is inclinedly extended from an outer peripheral edge toward a center, so as to extend toward the top opening of the collector 1 to guide the urine toward the container 1 through the top opening thereof. In other words, the slope of the top side of the top cover 13 is gradually decreased from an outer edge of the top cover 13 to an inner edge thereof. is Furthermore, the discharging unit 12 comprises a collecting port 121 located at the surrounding wall of the container 1 and a discharging port 122, wherein the collecting port 121 is operatively linked with the urine guider 11 and the discharging port 122 is operatively linked with the transporting tube 2, and the collecting port 121 and the discharging port 122 are operatively connected with each other. The discharging port 122 is connected to the outer wall of the transporting tube 2. In other words, the urine moves along the guider 11 to reach the collecting port 121, and passes through the collecting port 121 and the discharging port 122 respectively, so as to reach the transporting tube 2, and then the urine moves along the transporting tube 2 to be guide into the container 3.

Accordingly, the collector 1 is placed below a hip of the patient, especially female patient, wherein the collector serves as a temporary collecting station for guiding the urine to flow into the container 3.

FIG. 4 illustrates a second alternative mode the urine collector, wherein the urine collector comprises a collector 1 having a top opening and a cavity, a transporting tube 2, which is made of polycarbonate material, connected to the collector 1 for transporting the urine, and a container 3 operatively connected with the transporting tube 2 for containing the urine from the collector 1 through the transporting tube 2. The collector 1 is a pot-shaped collector and made of a rigid rubber material. Accordingly, the collector 1 comprises a urine guider 11 provided at a bottom portion of the collector 1 for guiding urine at the urine guider 11 within the cavity, and a discharging unit 12 operatively connected with the urine guider 11 of the collector 1, wherein the urine guider 11 is a guiding groove indented around a peripheral portion of the bottom wall of the collector 1. It is worth mentioning that a top portion of the urine guider 11 is lower than the bottom wall of the collector 1. And, the collector 1 further comprises a surrounding wall, wherein the surrounding wall is an inclined wall extended with respect to the bottom wall of the collector 1 that the size of the container 1 is gradually reduced from the top opening of the collector 1 to the bottom wall of the collector 1. In other words, the urine guider 11 is formed between the surrounding wall and the bottom wall of the collector 1.

Moreover, the collector 1 further comprises a ring-shaped top cover 13 coaxially coupled on a top edge of the collector 1 to partially cover the top opening thereof, wherein the top cover 13 is preferably made of latex. A top side of the top cover 13 is inclinedly extended from an outer peripheral edge toward a center, so as to extend toward the top opening of the collector 1 to guide the urine toward the container 1 through the top opening thereof. In other words, the slope of the top side of the top cover 13 is gradually decreased from an outer edge of the top cover 13 to an inner edge thereof. Furthermore, the discharging unit 12 comprises a collecting port 121 located at the surrounding wall of the container 1 and a discharging port 122, wherein the collecting port 121 is operatively linked with the urine guider 11 and the discharging port 122 is operatively linked with the transporting tube 2, and the collecting port 121 and the discharging port 122 are operatively connected with each other. The discharging port 122 is connected to the outer wall of the transporting tube 2. In other words, the urine moves along the guider 11 to reach the collecting port 121, and passes through the collecting port 121 and the discharging port 122 respectively, so as to reach the transporting tube 2, and then the urine moves along the transporting tube 2 to be guide into the container 3.

Accordingly, the collector 1 is placed below a hip of the patient, especially female patient, wherein the collector serves as a temporary collecting station for guiding the urine to flow into the container 3.

According to the above mentioned embodiment and its alternatives, the collector 1 can be made of polymer materials, such as rigid plastic, rigid rubber, or polycarbonate materials. In other words, since the collector 1 is made of light in weight materials, the collector 1 is easy to be carried, and it is convenient for the patients to use. In addition, the transporting tube 2 of the embodiment and its alternatives can be made of PET, silica gel, and latex, such that the above mentioned materials are light in weight and are durable.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A urine collector for collecting urine from a user, comprising:
a collector having a bottom wall and a surrounding wall upwardly extended therefrom to define a cavity;
a top cover coupled on top of said collector, wherein said top cover has a ring shape and defining a top side with a size adapted for covering a hip of the user, wherein said top side of said top cover is sloped downwardly from an outer peripheral edge of said top cover to an inner peripheral edge thereof for guiding urine flowing into said collector; and
a urine guider provided at a bottom portion of said collector for guiding urine at said urine guider within said cavity of said collector.

2. The urine collector, as recited in claim 1, wherein said urine guider has a guiding groove indentedly formed at said bottom wall of said collector.

3. The urine collector, as recited in claim 2, wherein said guiding groove is indented at a peripheral portion of said bottom wall of said collector.

4. The urine collector, as recited in claim 1, wherein a top portion of said urine guider is lower than said bottom wall of said collector.

5. The urine collector, as recited in claim 3, wherein a top portion of said urine guider is lower than said bottom wall of said collector.

6. The urine collector, as recited in claim 1, wherein said urine guider is formed between said surrounding wall and said bottom wall of said collector.

7. The urine collector, as recited in claim 5, wherein said urine guider is formed between said surrounding wall and said bottom wall of said collector.

8. The urine collector, as recited in claim 1, further comprising a discharging unit operatively connected with said urine guider and a container operatively linked to said discharging unit for collecting urine from said urine guider.

9. The urine collector, as recited in claim 3, further comprising a discharging unit operatively connected with said urine guider for discharging urine from said urine guider, and a container operatively linked to said discharging unit for collecting urine from said urine guider.

10. The urine collector, as recited in claim 7, further comprising a discharging unit operatively connected with said urine guider for discharging urine from said urine guider, and a container operatively linked to said discharging unit for collecting urine from said urine guider.

11. The urine collector, as recited in claim 8, further comprising a transporting tube extended to said container, wherein said discharging unit comprises a collecting port located at said surrounding wall of said container and a discharging port operatively linked to said collecting port, wherein said collecting port is operatively linked with said urine guider and said discharging port is operatively linked with said transporting tube for discharging urine from said urine guider to said container via said transporting tube.

12. The urine collector, as recited in claim 9, further comprising a transporting tube extended to said container, wherein said discharging unit comprises a collecting port located at said surrounding wall of said container and a discharging port operatively linked to said collecting port, wherein said collecting port is operatively linked with said urine guider and said discharging port is operatively linked with said transporting tube for discharging urine from said urine guider to said container via said transporting tube.

13. The urine collector, as recited in claim 10, further comprising a transporting tube extended to said container, wherein said discharging unit comprises a collecting port located at said surrounding wall of said container and a discharging port operatively linked to said collecting port, wherein said collecting port is operatively linked with said urine guider and said discharging port is operatively linked with said transporting tube for discharging urine from said urine guider to said container via said transporting tube.

14. The urine collector, as recited in claim 13, wherein said transporting tube is made of material selected from a group consisting of PET, rubber and polycarbonate.

15. The urine collector, as recited in claim 1, wherein said surrounding wall is a vertical wall extended with respect to said bottom wall of said collector that a size of said top opening of said collector matches a size of said bottom wall thereof.

16. The urine collector, as recited in claim 14, wherein said surrounding wall is a vertical wall extended with respect to said bottom wall of said collector that a size of said top opening of said collector matches a size of said bottom wall thereof.

17. The urine collector, as recited in claim 1, wherein said surrounding wall is an arc-shaped wall extended with respect to said bottom wall of said collector that a size of said top opening of said collector is larger than a size of said bottom wall thereof.

18. The urine collector, as recited in claim 14, wherein said surrounding wall is an arc-shaped wall extended with respect to said bottom wall of said collector that a size of said top opening of said collector is larger than a size of said bottom wall thereof.

19. The urine collector, as recited in claim 1, wherein said surrounding wall is an inclined wall extended with respect to said bottom wall of said collector that a size of said container is gradually reduced from said top opening of said collector to said bottom wall thereof.

20. The urine collector, as recited in claim 14, wherein said surrounding wall is an inclined wall extended with respect to said bottom wall of said collector that a size of said container is gradually reduced from said top opening of said collector to said bottom wall thereof.
